(19) United States
(12) Patent Application Publication
(10) Pub. No.: US 2020/0113846 A1
(43) Pub. Date: Apr. 16, 2020

(54) A TOPICAL OCULAR SUSPENSION OF ANTIOXIDANTS

(71) Applicant: Tathagata DUTTA, BERHAMPORE (IN)

(72) Inventor: Tathagata DUTTA, BERHAMPORE (IN)

(21) Appl. No.: 16/618,142

(22) PCT Filed: Mar. 26, 2018
(86) PCT No.: PCT/IN2018/050169

§ 371 Date: Nov. 28, 2019

(30) Foreign Application Priority Data
Jul. 25, 2017 (IN) 201741026301

(51) Int. Cl. A61K 31/047; A61K 47/38; A61K 47/36; A61K 9/00

(52) U.S. Cl. CPC A61K 31/047; A61K 9/0048; A61K 47/36; A61K 47/38

(57) ABSTRACT

A topical ocular suspension for treating or preventing age related macular degeneration (ARMD) comprising of one or more antioxidants and at least one pharmaceutically acceptable excipient, wherein the antioxidant is a carotenoid selected from lutein, zeaxanthin or a combination thereof.

### FIELD OF THE INVENTION

This invention relates to drug delivery dosage forms and methods to treat medical conditions of the eye. Specifically, this invention relates to suspension drug delivery dosage forms of antioxidants for drug delivery within the eye.

### BACKGROUND OF THE INVENTION

Age related macular degeneration (ARMD) is a severe problem of the eye which accounts for the loss of vision of huge number of elderly population across the globe. The disease which starts as a mild innocent problem of the eye, including occasional floaters and black dots in front of the eye gradually progresses to the loss of peripheral vision to complete loss of vision.

Antioxidants are well known to slow down the progression of the ARMD disease. There are several formulations available in the market containing single or combination of antioxidants like Alpha Tocopherol, Lutein, Zeaxanthin, Selenium, etc for oral administration for slowing down the progression of the disease. However, these orally administered anitioxidants suffers from several limitations, viz. the drug does not reach the eye at appropriate concentrations and has either none or very poor pharmacological action in the eye when administered through oral route.

Hence, there is a need for a topical formulation of antioxidants like Lutein and/or Zeaxanthin for ocular administration that will release the drug at a predetermined rate for an extended period of time thereby maintaining a steady concentration of antioxidants in the aqueous and vitreous humor thereby increasing the macular pigment optical density (MPOD), which will be helpful for better management of ARMD.

### SUMMARY OF THE INVENTION

Accordingly, the invention provides new topical drug delivery system that releases the active agent at a predetermined rate effective to sustain release of the said active agent to achieve the desired therapeutic effects, and methods of making such systems. The topical drug delivery system of the invention is an ocular suspension. The topical ocular suspension of the invention releases the drug at a predetermined rate for an extended period of time thereby maintaining a steady concentration of antioxidants in the aqueous and vitreous humor, thereby increasing the macular pigment optical density (MPOD).

The topical ocular suspension for treating or preventing age related macular degeneration (ARMD) described herein comprises one or more antioxidants and at least one pharmaceutically acceptable excipient. The antioxidant according to the disclosure herein is a carotenoid selected from lutein, zeaxanthin or a combination thereof. The concentration of lutein in the topical ocular suspension is about 0.03% to 6.0% w/v and the concentration of zeaxanthin in the topical ocular suspension is about 0.03% to 6%. The topical ocular suspension further comprises a suspending agent. The suspending agent is selected from the group comprising of cellulose derivatives, gums, Polyvinyl Alcohol (PVA), Povidone K30, Povidone K 90, Polycarbophil, Polyethylene Glycol (PEG), Carbomer or a combination thereof. The cellulose derivative is Hydroxypropyl methyl cellulose (HPMC), Hydroxypropyl cellulose (HPC), Methyl Cellulose, Carboxy methylcellulose sodium, Carboxyethyl cellulose sodium, Hydroxyethyl cellulose or a combination thereof. The gum is Gaur gum, Gellan gum, Xanthan gum or a combination thereof.

### DETAILED DESCRIPTION OF THE INVENTION

As described herein, the disclosure provides a topical ocular suspension that releases the drug at a predetermined rate for an extended period of time thereby maintaining a steady concentration of antioxidants in the aqueous and vitreous humor, thereby increasing the macular pigment optical density (MPOD).

The topical ocular suspension for treating or preventing age related macular degeneration (ARMD) described herein comprises one or more antioxidants and at least one pharmaceutically acceptable excipient. The antioxidant according to the disclosure herein is a carotenoid selected from lutein, zeaxanthin or a combination thereof. The concentration of lutein in the topical ocular suspension is about 0.03% to 6.0% w/v and the concentration of zeaxanthin in the topical ocular suspension is about 0.03% to 0.06%. The topical ocular suspension further comprises a suspending agent. The suspending agent is selected from the group comprising of cellulose derivatives, gums, Polyvinyl Alcohol (PVA), Povidone K30, Povidone K 90, Polycarbophil, Polyethylene Glycol (PEG), Carbomer or a combination thereof. The cellulose derivative is Hydroxypropyl methyl cellulose (HPMC), Hydroxypropyl cellulose (HPC), Methyl Cellulose, Carboxy methylcellulose sodium, Carboxyethyl cellulose sodium, Hydroxyethyl cellulose or a combination thereof. The gum is Gaur gum, Gellan gum, Xanthan gum or a combination thereof.

In one embodiment, the topical ocular suspension comprises the antioxidants lutein and zeaxanthin, a suspending agent, a preservative and pH adjusting agents.

### Example: 1

Lutein+Zeaxanthin Ocular Suspension

| S. No | Ingredient | mg/mL |
|---|---|---|
| 1 | Lutein | 0.03-6 mg |
| 2 | Zeaxanthin | 0.03-6 mg |
| 3 | Hydroxy Propyl Methyl Cellulose (HPMC) | 3 mg-9 mg |
| 4 | Benzalkonium Chloride | 0.001% |
| 5 | Water for Injection | q.s |
| 6 | Hydrochloride acid/sodium hydroxide | q.s |

Process: Required quantities of lutein and zeaxanthin are mixed with HPMC and Benzalkonium Chloride was added as a preservative and mixed with water for injection in a homogenizer. The suspension so formed is made up to the volume with water for injection and aseptically filled into containers, and sealed.

In another embodiment, the topical ocular suspension comprises the antioxidants lutein, a suspending agent, a preservative and pH adjusting agents.

### Example: 2

Lutein Ocular Suspension

| S. No | Ingredient | mg/mL |
|---|---|---|
| 1 | Lutein | 0.03-6 mg |
| 2 | Xanthan gum | 0.6% |
| 3. | Disodium Edetate | 0.13% |
| 4. | Phenyl Mercuric Nitrate | 0.002% |
| 5. | Sodium Chloride | 0.03% |
| 6. | Hydrochloric Acid/Sodium Hydroxide | q.s |
| 7. | Water for Injection | q.s |

Process: Required quantity of Lutein is mixed with Xanthan gum and Disodium EDTA. To the mixture so obtained, Phenyl Mercuric Nitrate was added as a preservative and sodium chloride as Osmolarity adjusting agent and mixed with water for injection in a homogenizer. The suspension so formed is made up to the volume with water for injection & the pH was adjusted using hydrochloric acid/sodium hydroxide and aseptically filled into containers, and sealed.

In another embodiment of the invention, the topical ocular suspension comprises zeaxanthin and pharmaceutical excipients.

### Example: 3

Zeaxanthin Ocular Suspension

| S. No | Ingredient | mg/mL |
|---|---|---|
| 1 | Zeaxanthin | 0.03-6 mg |
| 2 | Tyloxapol | 0.3% |
| 3 | Sodium citrate | 0.5-5 mg |
| 4 | Citric Acid | 0.3-1.5 mg |
| 5 | Benzalkonium chloride | 0.001% |
| 6 | Hydrochloric Acid/Sodium Hydroxide | q.s |
| 7 | Water for Injection | q.s |

Process: Required quantity of Zeaxanthin is mixed with Tyloxapol, sodium citrate & citric acid. To the mixture so obtained, Benzalkonium chloride was added as a preservative and mixed with water for injection in a high shear mixer. The suspension so formed is made up to the volume with water for injection & the pH was adjusted using hydrochloric acid/sodium hydroxide and aseptically filled into containers, and sealed.

### Example: 4

Lutein+Zeaxanthin Ocular Suspension

| S. No | Ingredient | mg/mL |
|---|---|---|
| 1 | Lutein | 0.03-6 mg |
| 2 | Zeaxanthin | 0.03-6 mg |
| 3 | Polyethylene Glycol (PEG) 400 | 1-5% |
| 4 | Polyethylene Glycol (PEG) 4000 | 3-9% |
| 5 | Sodium Chloride | 0.05% |
| 6 | Phenyl Mercuric Nitrate | 0.002% |
| 7 | Water for Injection | q.s |

Process: Required quantities of Lutein & Zeaxanthin were well mixed with PEG 4000, followed by addition of Phenyl mercuric nitrate & sodium chloride. To the mixture so obtained, was added required quantity of PEG 400, followed by addition of required quantity of water for Injection along with continuous stirring in a high shear mixer/homogenizer. The suspension so formed is filled aseptically and sealed.

### Example: 5

Lutein+Zeaxanthin Ocular Suspension

| S. No | Ingredient | mg/mL |
|---|---|---|
| 1 | Lutein | 0.03-6 mg |
| 2 | Zeaxanthin | 0.03-6 mg |
| 3 | Povidone K30 | 0.1-0.6% |
| 4 | Povidone K90 | 1.0-6% |
| 5 | Sodium Sulphate | 0.9 mg |
| 6 | Sodium Citrate | 0.2 mg |
| 7 | Citric Acid | 0.6 mg |
| 8 | Benzalkonium Chloride | 0.001% |
| 9 | Hydrochloric Acid/Sodium Hydroxide | q.s |
| 10 | Water for Injection | q.s |

Process: Required quantities of Lutein & Zeaxanthin were well mixed with Povidone K30 & Povidone K90, followed by addition of sodium citrate, citric acid & sodium sulphate. To the mixture so obtained, was added required quantity of Benzalkonium chloride, followed by addition of required quantity of water for Injection along with continuous stirring in a high shear mixer/homogenizer. The pH was adjusted using required quantities of sodium hydroxide/hydrochloric acid. The suspension so formed is filled aseptically and sealed.

### Example: 6

Lutein+Zeaxanthin Ocular Suspension

| S. No | Ingredient | mg/mL |
|---|---|---|
| 1 | Lutein | 0.03-6 mg |
| 2 | Zeaxanthin | 0.03-6 mg |
| 3 | Hydroxy Propyl Methyl | 0.03-30 mg |
| | Cellulose (HPMC) | |
| 4 | Hydroxypropyl Cellulose | 0.15-30 mg |
| | (HPC) | |
| 5. | Benzalkonium Chloride | 0.001% |
| 6. | Sodium Chloride | 0.01 mg |
| 7. | Hydrochloric Acid/Sodium | q.s |
| | Hydroxide | |
| 8. | Water for Injection | q.s |

Process: Required quantities of Lutein and Zeaxanthin are mixed with HPMC, HPC and Sodium Chloride and to this was added Benzalkonium Chloride and Water for Injection. The suspension so formed was mixed in a homogenizer and the pH was adjusted using hydrochloric acid or sodium hydroxide and the volume was made adjusted using water for injection and filled and sealed.

The method for preparing the topical ocular suspension according to the disclosure described herein is not limited to the above methods. The topical ocular suspension according to the disclosure described herein can be prepared by using various other techniques.

We claim:

1. A topical ocular suspension for treating or preventing age related macular degeneration (ARMD) comprising of one or more antioxidants and at least one pharmaceutically acceptable excipient, wherein the antioxidant is a carotenoid selected from lutein, zeaxanthin or a combination thereof.

2. The topical ocular suspension of claim 1, wherein the concentration of lutein is about 0.03% to 6.0% w/v.

3. The topical ocular suspension of claim 1, wherein the concentration of zeaxanthin is about 0.03% to 6.0% w/v.

4. The topical ocular suspension of claim 1 further comprises a suspending agent.

5. The topical ocular suspension of claim 1 wherein the suspending agent is a cellulose derivative, gum, Polyvinyl Alcohol (PVA), Povidone K30, Povidone K 90, Polycarbophil, Polyethylene Glycol (PEG), Carbomer or a combination thereof.

6. The topical ocular suspension of claim 1 wherein the cellulose derivative is Hydroxypropyl methyl cellulose (HPMC), Hydroxypropyl cellulose (HPC), Methyl Cellulose, Carboxy methylcellulose sodium, Carboxyethyl cellulose sodium, Hydroxyethyl cellulose or a combination thereof.

7. The topical ocular suspension of claim 1 wherein the gum is Gaur gum, Gellan gum, Xanthan gum or a combination thereof.
